(19) United States
(12) Patent Application Publication
(10) Pub. No.: US 2024/0324950 A1
(43) Pub. Date: Oct. 3, 2024

(54) Systems and Methods for Remote Patient Screening and Triage

(71) Applicant: Sleep Number Corporation, Minneapolis, MN (US)

(72) Inventors: Omid Sayadi, San Jose, CA (US); Steven Jay Young, Los Gatos, CA (US); Carl Hewitt, San Jose, CA (US)

(21) Appl. No.: 18/398,611

(22) Filed: Dec. 28, 2023

Related U.S. Application Data
(63) Continuation of application No. 17/112,074, filed on Dec. 4, 2020, now abandoned.
(60) Provisional application No. 63/003,551, filed on Apr. 1, 2020.

(51) Int. Cl. A61B 5/00; A61B 5/0205; A61B 5/024; A61B 5/08; A61B 5/11; G06F 21/60; G10L 15/18; G10L 15/22; G10L 25/66; G10L 25/78; G16H 40/67

(52) U.S. Cl. CPC A61B 5/4818; A61B 5/0022; A61B 5/0205; A61B 5/0823; A61B 5/11; A61B 5/4842; A61B 5/6887; A61B 5/7405; A61B 5/746; A61B 5/747; A61B 5/749; G06F 21/602; G10L 15/1815; G10L 15/22; G10L 25/66; G10L 25/78; G16H 40/67; A61B 5/024; A61B 5/0816; A61B 5/7203; A61B 5/7267; A61B 2562/0204; A61B 2562/0219; A61B 2562/0247; G10L 2015/223

(57) ABSTRACT

A system for short-term screening and triage of a subject includes a smart device in the subject's possession and an application on the smart device programmed to provide instructions to the subject to start a screening procedure, the application programmed with multiple screening procedures, and record sensor data obtained from a sensor located in the smart device.

### CROSS-REFERENCE TO RELATED APPLICATIONS

This application is a continuation of U.S. application Ser. No. 17/112,074, filed Dec. 4, 2020, which claims priority to and the benefit of U.S. Provisional Application Patent Ser. No. 63/003,551, filed Apr. 1, 2020, the entire disclosure of which is hereby incorporated by reference.

### TECHNICAL FIELD

This disclosure relates to remote biosignal monitoring of a subject, cardiac, and respiration monitoring being non-limiting examples. More particularly, the disclosure pertains to systems and methods for short-term and long-term patient screening with symptoms of a disease, bacterial or viral infections, cardiac related complications or respiratory related complications as examples. The disclosure further relates to using such systems to triage patients in order to determine the risk level and priority for further evaluation.

### BACKGROUND

Traditionally, an office or clinic visit is required for monitoring, diagnosis and evaluation of patients who might present symptoms of infection, or cardio-respiratory complications. This process risks increased exposure of the public as well as the clinical team and may overload the in-patient hospital system capacity at times of crisis or pandemic in addition soldiers in the field may not have caregivers in the vicinity. Currently, there are too many patients that require monitoring for the existing healthcare infrastructure but there are no high volume accurate remote monitoring tools available that can be easily used or deployed without physical access to a caregiver. Unlike persistent condition, paroxysmal conditions with sudden or intermittent onset require an at home screening solution that can be used immediately and continuously.

### SUMMARY

Disclosed herein are systems that use optical, audio, radio, sensors such as accelerometer, gyroscope, pressure, load, weight, force, motion or vibration to capture the mechanical vibrations of the body as well as physiological movements of heart and lungs and translate that into biosignal information that can be used for screening and identifying disease conditions. The systems and methods presented here can be used by a subject when experiencing symptoms of a complication or condition, or can be used when instructed by a physician in a telehealth application.

The systems are used for short-term and long-term screening. Short-term screening systems can include cell phones, tablets, wearable watches or any accessory available to the patient that has one or more sensors that can capture the mechanical vibrations of the body, heart and lungs such as accelerometer, gyroscope, pressure, load, weight, force, motion or vibration. Such devices can be placed near the source of body's physiological sources such as heart and lung, including but not limited to placement on the chest, abdomen, side, back, or the like. Long-term screening systems can include installable sensors into the legs or under the legs of the bed, which can capture the mechanical vibrations of the body, heart and lungs such as accelerometer, gyroscope, pressure, load, weight, force, motion or vibration. Short-term screening is intended for limited duration tests (seconds to minutes) whereas long-term screening can be used continuously for any duration (seconds, minutes, days, months, etc). The short-term and long-term screening systems can work independently or can be synchronized and work in unison to exchange data, for example, subject's historical trend data or baseline data.

The systems can be used as a patient triage tool to help assess the degree of urgency. The systems can include a smart phone app installation to enable screening and evaluating the patient's status.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIGS. 1A-1D illustrate an example system for short-term screening and triage using a subject's smart device, as disclosed herein.

FIG. 1E is a set of data streams recorded during a short-term screening using a subject's smart device, as disclosed herein.

FIG. 2A is a flow diagram of another example system for short-term screening and triage as disclosed herein.

FIG. 2B is a flow diagram of an example system for short-term and long term screening and triage as disclosed herein.

FIG. 2C is a system architecture for implementing short-term and long term screening and triage.

FIG. 3 is a flow diagram of an example process to collect sensor data.

FIG. 4 is a flow diagram of an example process for short-term analysis of sensor data.

FIG. 5 is a flow diagram of an example process for short-term cardiac analysis.

FIG. 6 is a flow diagram of an example process for short-term respiratory analysis.

FIG. 7 is a flow diagram of an example process for short-term coughing analysis.

FIG. 8 is a flow diagram of an example process for short-term screening and triage based on machine learning classifiers.

### DETAILED DESCRIPTION

Methods are disclosed to develop remote screening procedures and use sensor data from such systems to triage a subject's health status. In implementations, the systems can analyze cardiac information of the subject and determine cardiac rhythm, morphology, and rate information. The cardiac rhythm, morphology, and rate information can be used to identify the start or worsening of a cardiac condition such as atrial fibrillation, atrial flutter, ventricular fibrillation, ventricular flutter, bundle branch blocks, valve stenosis, myocardial ischemia, and supraventricular tachycardia. In implementations, the systems can analyze respiratory information of the subject and determine respiration rhythm and rate information. The respiration rhythm and rate information can be used to identify start or worsening of a respiratory condition such as shortness of breath, apnea or hypopnea. In implementations, the systems can analyze coughing information of the subject and determine coughing rhythm and rate information. The coughing rhythm and rate information can be used to identify the start or worsening of a coughing condition or breathing flow such as wheezing, rales, snoring and rhonchi. The systems can determine the severity of the condition, and/or changes and trends of the condition. The systems can generate remote screening reports. The systems can collect the data and the generated report can be stored, sent to a physician, be accessed by the physician for review, or analyzed using AI techniques.

In implementations, the screening system can be used to monitor the body's response to bacterial or viral infection. In particular, the sensors data can be used to monitor symptoms which may be directly or indirectly related to the increase in the body's temperature. Such symptoms can include changes in respiration flow and depth, respiration rate, heart rate, heart rate variability, movement and agitation, weight, and fluid retention. The system can further create a baseline for the patient, and continuously track and spot these changes over time, helping them become aware of their body's immune system response and enabling them, or a caregiver, to monitor their sickness.

In implementations, the screening system can be used in conjunction with a ventilator to remotely monitor the ventilator's efficacy. In addition, the system can be used with other sensors as a data hub to relay local data from pulse oximeters, thermometers, blood pressure or other sensors to a cloud enabling remote monitoring of these additional sensors for enhanced screening.

In implementations, the system will include bi-directional audio, text, and video to communicate with the patient.

In implementations, the screening system can also be used to screen for cardiovascular and autonomic indices. For example, the system can be used for in home stress testing where sensors data can be used to monitor indices of heart rate variability to quantify dynamic autonomic modulation or heart rate recovery.

In implementations, the system can be used to create events based on the vital's analysis. The event may be an audible tone or message sent to the cloud for a critical condition. In implementations, the system enables data convergence between the short-term and long-term monitoring systems such that, for example, one system can use the historical data collected by the other system to establish a baseline, or to use such information to determine the progression of a disease or worsening of a condition.

In implementations, additional bed-based sensor data can be combined to remove or cancel out common mode or other noise sources. Mobile data acquisition sensors can be used in conjunction with other monitoring systems that have a fixed location so that data from different sources of data monitoring may be combined to increase total monitoring coverage when someone is mobile.

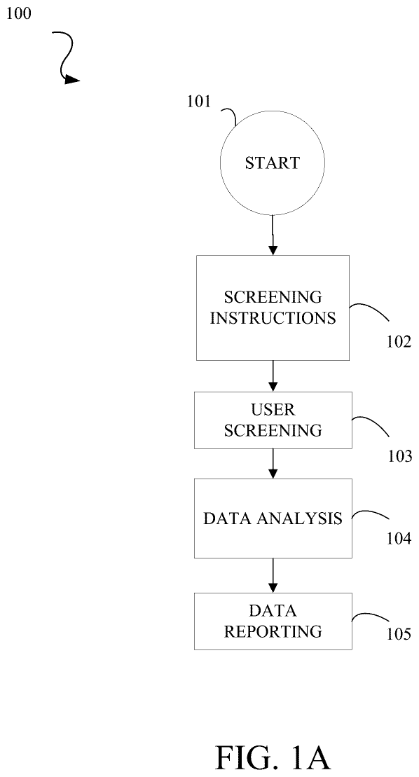

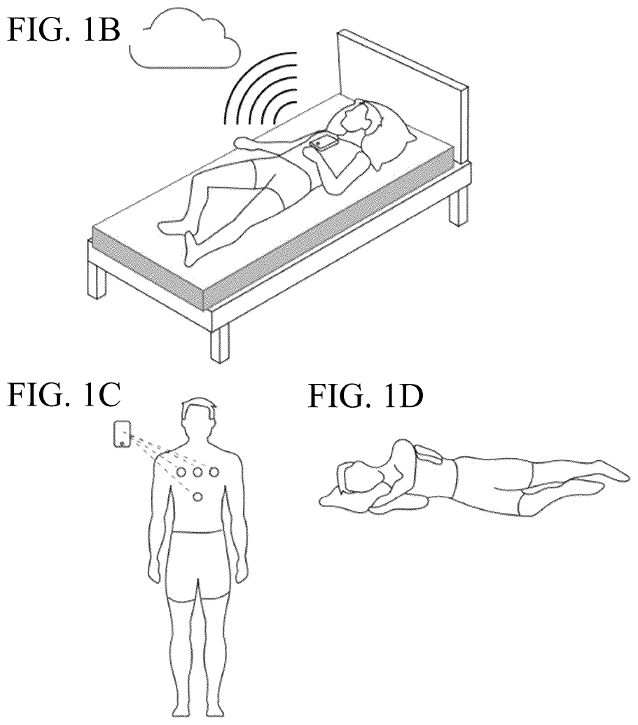

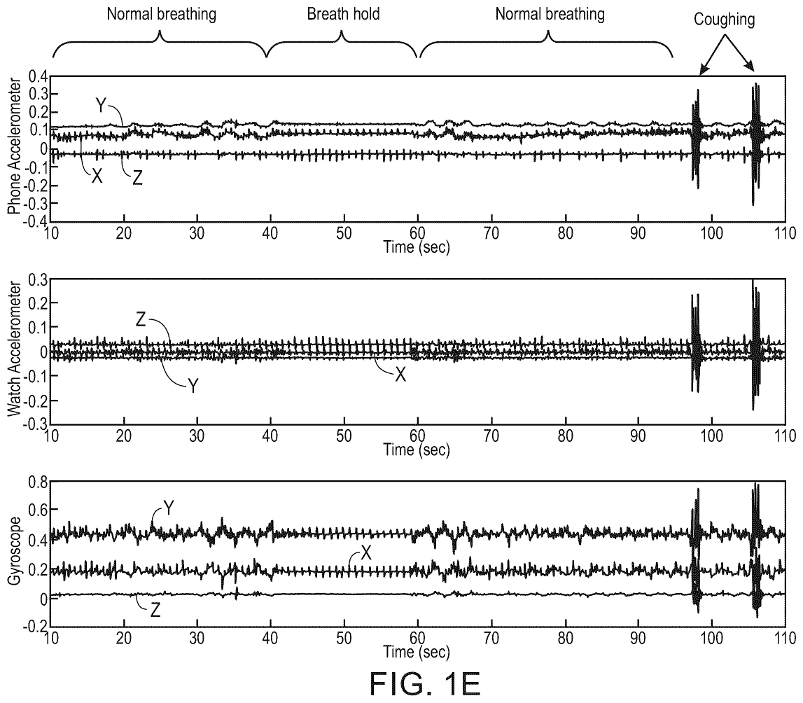

FIGS. 1A-1D illustrate an example system for short-term screening and triage using a subject's smart device. FIG. 1A is a flow chart of an example method 100 for using a screening and triage system using a subject's smart device. In implementations, the smart device can include cell phones, tablets, wearable watches, or any accessory available to the subject that has one or more sensors that can capture the mechanical vibrations of the body, heart, and lungs such as an accelerometer, gyroscope, pressure sensor, load sensor, weight sensor, force sensor, motion sensor, or vibration sensor. The figures illustrate a cell phone as a non-limiting example.

A start of the screening can be triggered by the start of the symptoms of an illness or condition (for example, when the subject is not feeling well) or can be initiated per the physician's request (101). The subject will receive screening instructions (102). In implementations, this can be given by the physician. In implementations, the instructions can be provided by an app installed on the smartphone or other mobile device. Instructions are condition specific; therefore, the screening procedure for an infection may be different from the screening procedure for a cardiac condition. FIGS. 1B-1D are example of screening instructions (103) where an app instructs the subject to perform screening procedures. In implementations, the app can instruct the subject to lay in bed, remain stationary for a given time (a counter can be used to display the time or to play a countdown tone) as shown in FIG. 1B. In implementations, the app can instruct the subject to place the phone on his/her chest at different locations as shown in FIG. 1C. In implementations, the app can instruct the subject to lay on his/her side as shown in FIG. 1D. In implementations, other placements are possible, for example on the back, stomach, abdomen, and the like. The app records the sensors data, analyzes the sensors data (104) and generates a health report (105). In implementations, the report can include physiological measurements, for example, heart rate, respiration rate, heart rate variability, etc. In implementations, the report can further include a list of identified or suspected issues, and a degree of urgency (severity level). In implementations, the app can send the data to the physician or caregiver, or can suggest follow-up tests using the same or a different system.

FIG. 1E are graphs of data streams recorded during a short-term screening using a subject's smart device. FIG. 1E shows example data streams when the subject is laying in bed, with his smartphone placed on his chest (as shown in FIG. 1B) and a smart watch worn on his wrist. The (X, Y, Z) data streams from the phone's accelerometer are plotted in the top panel. The (X, Y, Z) data streams from the watch's accelerometer are plotted in the mid panel. The (X, Y, Z) data streams from the phone's gyroscope are plotted in the bottom panel. The phone's accelerometer data captures both cardiac and respiration activity. In this example, the respiration signal is strongly seen in X and Y components whereas the heart activity is strongest in Z direction. The watch's accelerometer data doesn't capture the respiration signal. The watch only captures the cardiac activity. The gyroscope data captures both respiration and cardiac activity. The impact of a breath hold is visible in data streams where respiration is recorded (phone accelerometer and gyroscope). The coughing episodes are visible in all recorded data streams.

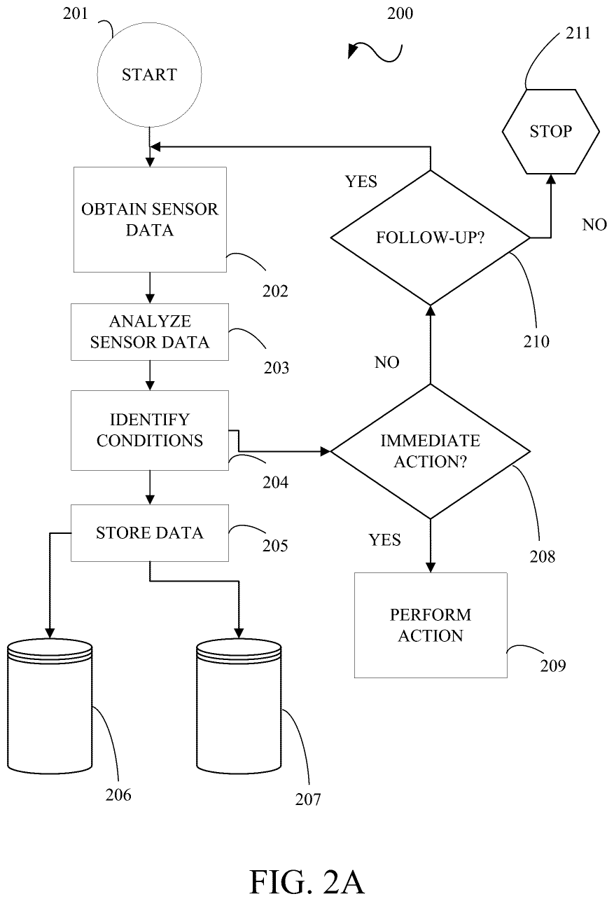

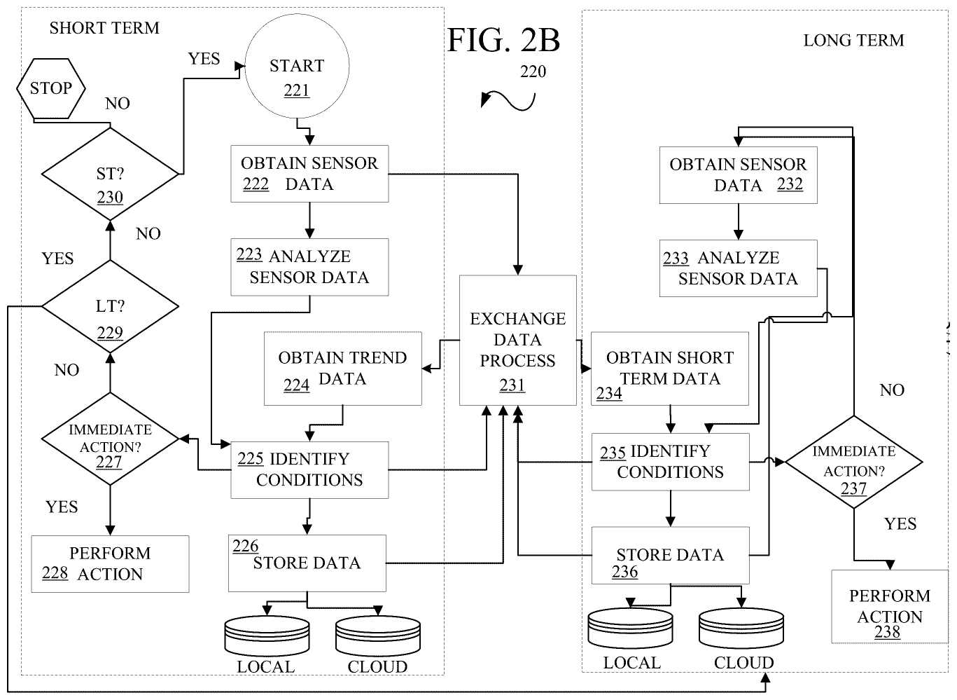

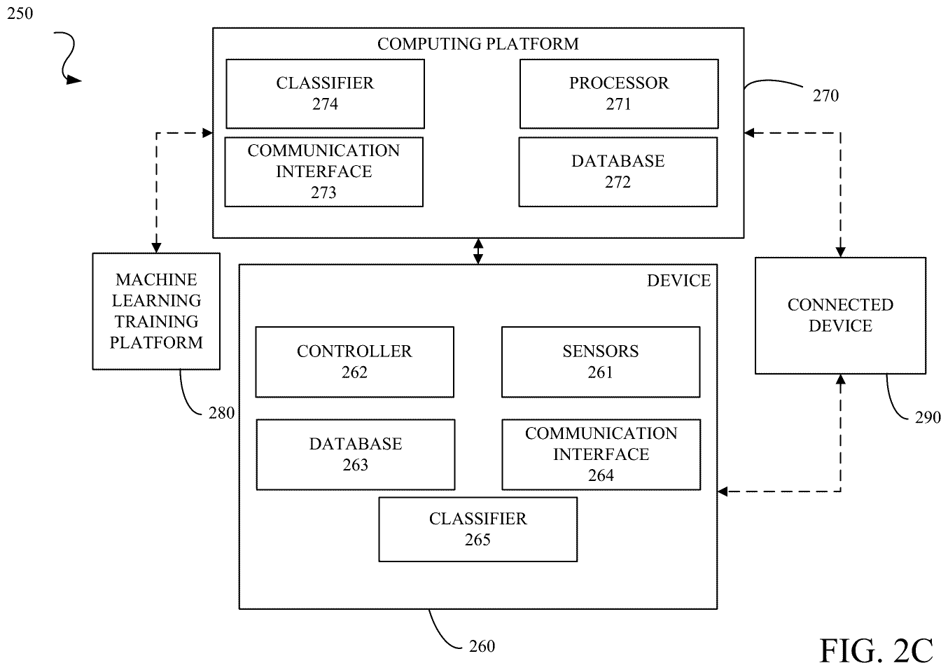

FIG. 2A is a flow diagram of an example of a method 200 for short-term screening and triage using the subject's smart device. In implementations, the smart device can include cell phones, tablets, wearable watches or any accessory available to the patient that has one or more sensors that can capture the mechanical vibrations of the body, heart and lungs such as accelerometer, gyroscope, pressure, load, weight, force, motion or vibration.

A start of the screening can be triggered by the start of the symptoms of an illness or condition (for example, when the subject is not feeling well) or can be initiated per the physician's request (201). After the screening starts, sensor data is obtained from the sensors (202). The sensor data is analyzed (203). Subject's conditions are identified using the analyzed metrics (204). In implementations, to quantify a body's response to bacterial or viral infection, changes in respiration flow and depth, respiration rate, heart rate, heart rate variability, movement and agitation, weight, and fluid retention are analyzed. Values outside a normal range may define an out-of-the-norm condition or if the system has access to a baseline for the patient, abrupt changes compared to the baseline may be detected as an out-of-the-norm condition. In implementations, normal range or out-of-the norm condition can be relative to data representing a general population. Once a condition has been identified, a determination is made as to whether immediate action is needed (208). An immediate action is taken if necessary (209). In implementations, immediate action can be a notification to the patient, a notification to the patient's physician, a call to a health center, or the like. If no immediate action is required, a determination is made as to whether a follow-up test is required to confirm the results or to provide new insights (210). If a follow-up is needed, additional or new sensor data is collected and the process starts anew. Otherwise, the screening process is terminated (211). In implementations, the sensors data, analyzed data, and identified data can be stored locally in a local database 206 or in a cloud database 207 for future access (205).

FIG. 2B shows an example method 220 for short-term and long-term screening and triage. Short-term screening can use subject's smart device. In implementations, the smart device can include cell phones, tablets, wearable watches or any accessory available to the patient that has one or more sensors that can capture the mechanical vibrations of the body, heart and lungs such as accelerometer, gyroscope, pressure, load, weight, force, motion or vibration. Long-term screening can use installable sensors into the legs or under the legs of a bed (an example of a substrate on which a subject can be positioned on), which can capture the mechanical vibrations of the body, heart and lungs such as accelerometer, gyroscope, pressure, load, weight, force, motion, or vibration. If the short-term screening determines that a long-term screening is required (229), the system can recommend adding a long-term screening system. The long-term screening can be added for enhanced continuous biosignal tracking.

Exchange data process 231 enables data exchange between the short-term and long-term screening, where trend data (i.e. subject's baseline data and historical data) can be accessed by either process. The exchange data process 231 also enables the long-term screening to have access to the short-term screening sensors data to be synchronized and added to the data stream sets for enhanced monitoring. In implementations, data from obtain sensor data 222, obtain sensor data 232, store data 226 including the local and cloud based storages, and store data 236 including the local and cloud based storages can be input into the exchange data process 231. In implementations, the exchange data process 231 outputs data to obtain trend data 224 and obtain trend data 234. That is, both initial and processed short-term data and long-term data can be exchanged between the short-term and long-term screening.

FIG. 2C is a system 250 and system architecture for implementing short-term and long term screening and triage. The system 250 includes one or more devices 260 which are connected to or in communication with (collectively “connected to”) a computing platform 270. In an implementation, a machine learning training platform 280 may be connected to the computing platform 270. In an implementation, users may access the data via a connected device 290, which may receive data from the computing platform 270 or the device 260. The connections between the one or more devices 260, the computing platform 270, the machine learning training platform 280, and the connected device 290 can be wired, wireless, optical, combinations thereof and/or the like. The system 250 is illustrative and may include additional, fewer or different devices, entities and the like which may be similarly or differently architected without departing from the scope of the specification and claims herein. Moreover, the illustrated devices may perform other functions without departing from the scope of the specification and claims herein.

In implementations, the system 250, the sensors and the data processing, for example, can be as described in U.S. patent application Ser. No. 16/777,385, filed on Jan. 30, 2020, U.S. patent application Ser. No. 16/595,848, filed Oct. 8, 2019, and U.S. Provisional Application Patent Ser. No. 62/804,623, filed Feb. 12, 2019 (collectively the “Applications”) the entire disclosures of which are hereby incorporated by reference.

In implementations, the device 260 can include one or more sensors 261, a controller 262, a database 263, and a communications interface 264. In an implementation, the device 261 can include a classifier 265 for applicable and appropriate machine learning techniques as described herein. The one or more sensors 261 can detect and capture vibration, pressure, force, weight, presence, and motion sensor data related to a subject.

In implementations, the controller 262 can apply the processes and algorithms described herein with respect to FIGS. 1A, 2A, 2B, and 4-8 to the sensor data to determine short-term and long-term screening biosignal information and data as described herein. In implementations, the classifier 265 can apply the processes and algorithms described herein with respect to FIGS. 1A, 2A, 2B, and 4-8 to the sensor data to determine short-term and long-term screening biosignal information and data. In implementations, the classifier 265 may be implemented by the controller 262. In implementations, the captured sensor data and the short-term and long-term screening biosignal information and data can be stored in the database 263. In an implementation, the captured sensor data and the short-term and long-term screening biosignal information and data can be transmitted or sent via the communication interface 264 to the computing platform 270 for processing, storage, and/or combinations thereof. The communication interface 264 can be any interface and use any communications protocol to communicate or transfer data between origin and destination endpoints. In an implementation, the device 260 can be any platform or structure which uses the one or more sensors 261 to collect the data from a subject(s) for use by the controller 262 and/or computing platform 270 as described herein. The device 260 and the elements therein may include other elements which may be desirable or necessary to implement the devices, systems, and methods described herein. However, because such elements and steps are well known in the art, and because they do not facilitate a better understanding of the disclosed embodiments, a discussion of such elements and steps may not be provided herein.

In implementations, the computing platform 270 can include a processor 271, a database 272, and a communication interface 273. In implementations, the computing platform 270 may include a classifier 274 for applicable and appropriate machine learning techniques as described herein. The processor 271 can obtain the sensor data from the sensors 261 or the controller 262 and can apply the processes and algorithms described herein with respect to FIGS. 1A, 2A, 2B, and 4-8 to the sensor data to determine short-term and long-term screening biosignal information and data as described herein. In an implementation, the processor 271 can obtain the short-term and long-term screening biosignal information and data as described herein from the controller 262 to store in database 272 for temporal and other types of analysis. In an implementation, the classifier 274 can apply the processes and algorithms described herein with respect to FIGS. 1A, 2A, 2B, and 4-8 to the sensor data to determine short-term and long-term screening biosignal information and data as described herein. The classifier 274 can apply classifiers to the sensor data to determine short-term and long-term screening biosignal information and data as described herein via machine learning. In an implementation, the classifier 274 may be implemented by the processor 271. In an implementation, the captured sensor data and the short-term and long-term screening biosignal information and data can be stored in the database 272. The communication interface 273 can be any interface and use any communications protocol to communicate or transfer data between origin and destination endpoints. In an implementation, the computing platform 270 may be a cloud-based platform. In an implementation, the processor 271 can be a cloud-based computer or an off-site controller. The computing platform 270 and elements therein may include other elements which may be desirable or necessary to implement the devices, systems, and methods described herein. However, because such elements and steps are well known in the art, and because they do not facilitate a better understanding of the disclosed embodiments, a discussion of such elements and steps may not be provided herein.

In an implementation, the machine learning training platform 280 can access and process sensor data to train and generate classifiers. The classifiers can be transmitted or sent to the classifier 265 or to the classifier 274.

In FIG. 2B, sensor data is obtained from sensors (232). In implementations, the sensor data can be analyzed, for example, similarly as shown in the Applications (233). Instantaneous or near instantaneous data from short term processing via exchange data process 231 is obtained by the long term processing (234). Conditions related to the subject are identified using the analyzed data and the obtained data (235). The identified conditions and data are stored in local or cloud based storage (236). As stated herein, the identified conditions and data are also input the exchange data process 231. A determination is made as to whether the identified conditions require immediate action (237). If not, nominal long term processing continues. If immediate action is needed, then a responsive action is performed (238).

In FIG. 2B, sensor data is obtained from sensors (221). In implementations, the sensor data can be analyzed, for example, similarly as shown in the Applications (223). Trend data from long term processing via exchange data process 231 is obtained by the short term processing (224). Conditions related to the subject are identified using the analyzed data and the obtained data (225). The identified conditions and data are stored in local or cloud based storage (226). As stated herein, the identified conditions and data are also input the exchange data process 231. A determination is made as to whether the identified conditions require immediate action (227). If immediate action is needed, then a responsive action is performed (228). If not needed, determine if long term processing is needed (229). If yes, perform long term processing. If not needed, determine if short term processing is needed (230). If not needed, current short term processing terminates. If yes, obtain data and perform another short term processing.

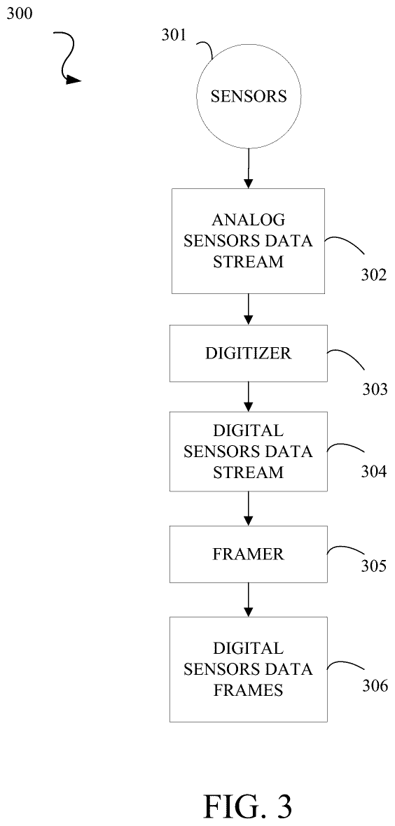

FIG. 3 is a processing pipeline 300 for obtaining sensor data such as, but not limited to, accelerometer, gyroscope, pressure, load, weight, force, motion or vibration. An analog sensors data stream 302 is received from the sensors 301. A digitizer 303 digitizes the analog sensors data stream into a digital sensors data stream 304. A framer 305 generates digital sensors data frames 306 from the digital sensors data stream 304 which includes all the digital sensors data stream values within a fixed or adaptive time window. The processing pipeline 300 shown in FIG. 3 is illustrative and can include any, all, none or a combination of the blocks or modules shown in FIG. 3. The processing order shown in FIG. 3 is illustrative and the processing order may vary without departing from the scope of the specification or claims.

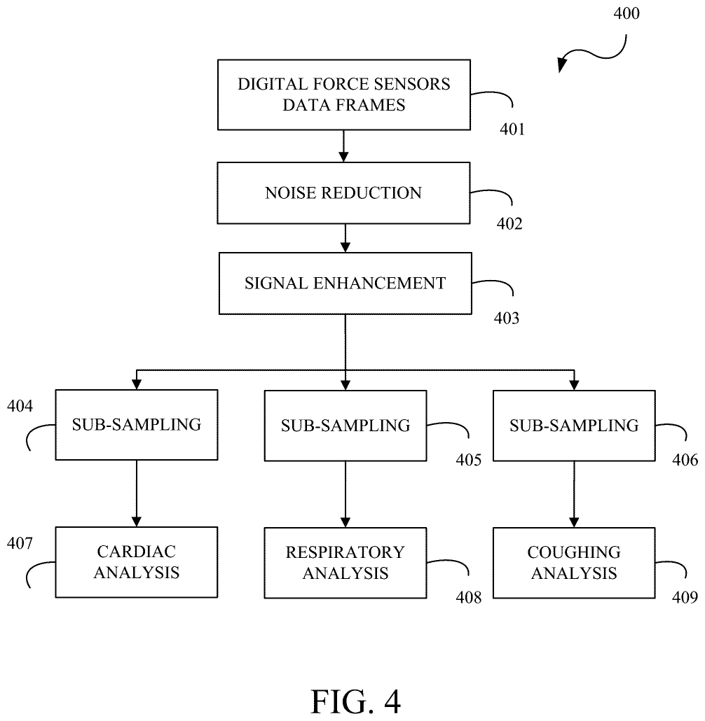

FIG. 4 is a pre-processing pipeline 400 for processing the sensor data. The pre-processing pipeline 400 processes digital sensor data frames 401. A noise reduction unit 402 removes or attenuates noise sources that might have the same or different level of impact on each sensor. The noise reduction unit 402 can use a variety of techniques including, but not limited to, subtraction, combination of the input data frames, adaptive filtering, wavelet transform, independent component analysis, principal component analysis, and/or other linear or nonlinear transforms. A signal enhancement unit 403 can improve the signal to noise ratio of the input data. The signal enhancement unit 403 can be implemented as a linear or nonlinear combination of input data frames. For example, the signal enhancement unit 403 may combine the signal deltas to increase the signal strength for higher resolution algorithmic analysis. Subsampling units 404, 405 and 406 sample the digital enhanced sensor data and can include downsampling, upsampling or resampling. The subsampling can be implemented as a multi-stage sampling or multi-phase sampling, and can use the same or different sampling rates for cardiac 407, respiratory 408, and coughing 409 analysis. The processing order shown in FIG. 4 is illustrative and the processing order may vary without departing from the scope of the specification or claims.

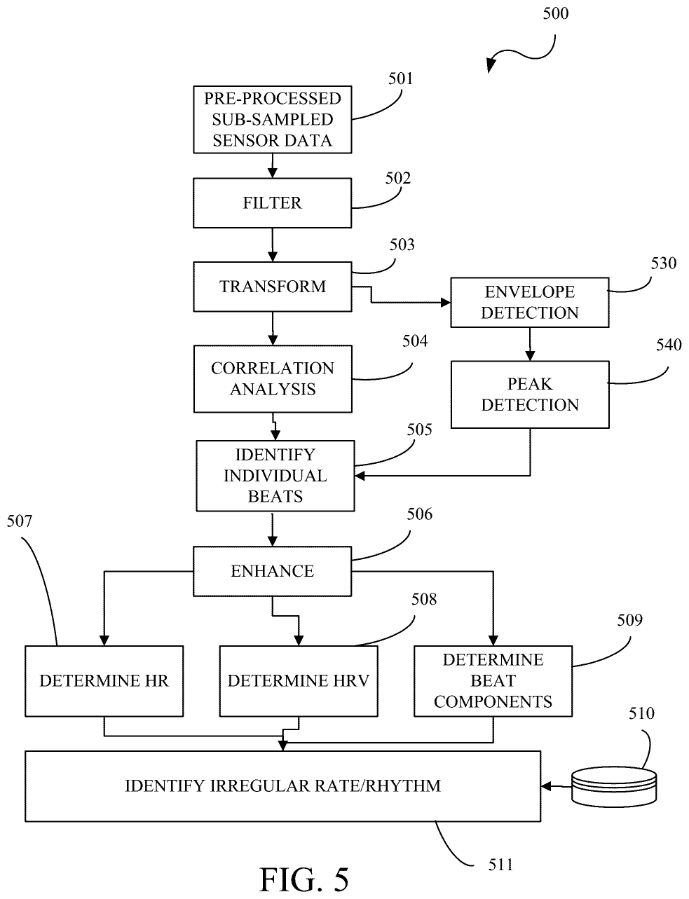

FIG. 5 is an example process 500 for cardiac analysis 407 using the pre-processed and sub-sampled data 501. Filtering is used to remove unwanted components of the input sensor data or to keep contents that are useful for cardiac processing (502). In implementations, the filtering can an infinite impulse response (IIR) filter, finite impulse response (IIR) filter, or a combination thereof. Filter can be low pass, high pass, bandpass, bandstop, notch or a combination of these. In implementations, the filtering can include sources from other sensors to remove common mode or other noise and can use adaptive filtering techniques to remove unwanted signals. The filtered sensor data is transformed to enhance cardiac components by modeling the input signal as a collection of waveforms of a particular form (sinusoids for the Fourier transform, mother wavelets for the wavelet transforms, and/or periodic basis functions for the periodicity transforms) (503). In implementations, the process can be a Fourier transform, wavelet transform, cosine transform or a math operation such as root-mean-square, absolute, moving average, moving median, etc.

Envelope detection is performed on the transformed sensor data, which takes a relatively high-frequency amplitude modulated signal as input and provides an output which is equivalent to the outline of the input data described by connecting all the local peaks in this signal (512). In implementations, envelope detection can use a low pass filter, a Hilbert transform or other envelope detection methods. Peak detection is performed to find local maximum and minimum points of the input signal (513). In implementations, peak detection can return all peaks, valleys or only the most dominant ones.

Correlation analysis is performed to measure the strength of relationship between different segments of the input signal using linear and nonlinear methods (504). The correlation analysis and peak locations can be used to identify individual beats in the input signal (505). The identified individual beats are enhanced (506). In implementations, this can include applying a window, a factor, or a transform to enhance specific characteristics of the signal. Time domain, frequency domain, or time frequency domain analysis can be performed to determine the heart rate using the enhanced individual beats (507). Time domain, frequency domain, or time frequency domain analysis can be performed to determine the heart rate variability metrics using the enhanced individual beats (508). In implementations, the heart rate variability metrics can include SDNN, RMSSD, PNN50, LF, HF and LF/HF indices. Time domain, frequency domain, or time frequency domain analysis can be performed to determine heartbeat components (509). For the cardiac signal, beat components can be P, Q, R, S and T waveforms, or atria/ventricular depolarization and repolarization. Irregular rate or rhythm can be detected in the cardiac data using the HR, HRV, beat components, and subject's trend data 510 (i.e. baseline and historical data) (511).

The processing order shown in FIG. 5 is illustrative and the processing order may vary without departing from the scope of the specification or claims.

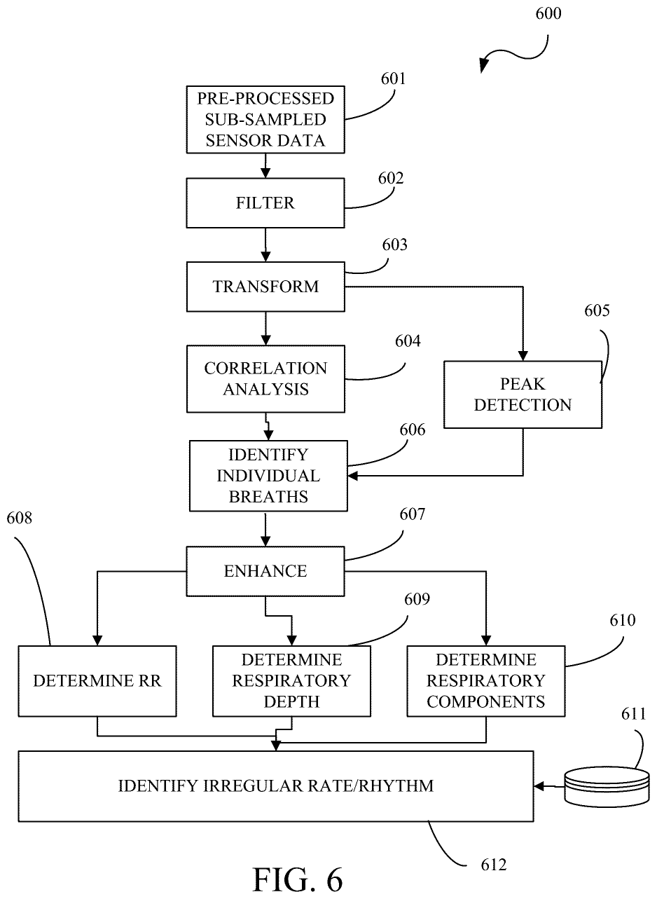

FIG. 6 is an example process for respiratory analysis 408 using the pre-processed and sub-sampled data 601. Filtering is used to remove unwanted components of the input sensor data or to keep contents that are useful for respiration processing (602). In implementations, the filter can use an IIR, FIR, or a combination thereof. In implementations, the filter can be low pass, high pass, bandpass, bandstop, notch, or a combination thereof. In implementations, the filter may include sources from other sensors to remove common mode or other noise and can use adaptive filtering techniques to remove unwanted signals. The filtered data is transformed to enhance respiratory components by modeling the input signal as a collection of waveforms of a particular form (sinusoids for the Fourier transform, mother wavelets for the wavelet transforms, periodic basis functions for the periodicity transforms) (603). The transform can be a Fourier transform, wavelet transform, cosine transform, or a math operation such as root-mean-square, absolute, moving average, moving median, etc. Peak detection is performed to find local maximum and minimum points of the input signal (605). In implementations, the peak detection can return all peaks, valleys, or only the most dominant ones.

Correlation analysis measures the strength of relationship between different segments of the input signal using linear and nonlinear methods (604). The correlation analysis and peak locations can be used to identify individual breaths in the input signal (606). The identified individual beats are enhanced (607). In implementations, this can include applying a window, or a factor, or a transform to enhance specific characteristics of the signal.

Time domain, frequency domain, or time frequency domain analysis can be used to determine the respiration rate using the enhanced individual breaths (608). Time domain, frequency domain, or time frequency domain analysis can be used to determine the respiration rate variability metrics using the enhanced individual breaths (609). In implementation, the respiration rate variability metrics can include DNN, RMSSD, PNN50, LF, HF and LF/HF indices. Time domain, frequency domain, or time frequency domain analysis can be used to determine breath components (610). For a respiratory signal, breath components can be inhale (inspiration) and exhale (expiration). Irregular rate or rhythm can be identified in the respiration data using the RR, RRV, breath components and subject's trend data 611 (i.e. baseline and historical data) (612).

The processing order shown in FIG. 6 is illustrative and the processing order may vary without departing from the scope of the specification or claims.

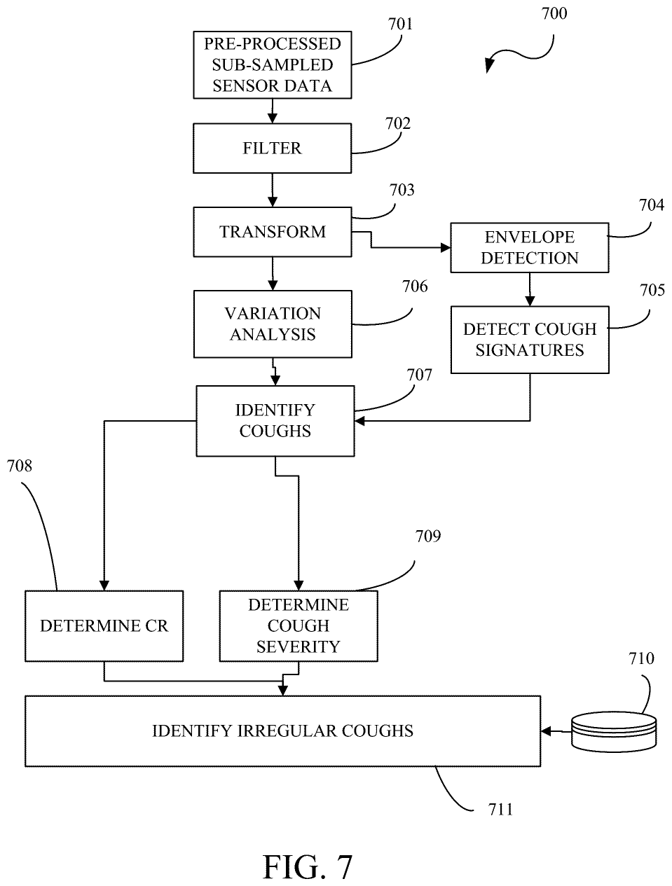

FIG. 7 is an example process for coughing analysis 409 using the pre-processed and sub-sampled data 701. Filtering is used to remove unwanted components of the input sensor data or to keep contents that are useful for coughing processing (702). In implementations, the filter can be an IIR, FIR or a combination of the two. In implementations, the filter can be low pass, high pass, bandpass, bandstop, notch, or a combination thereof. In implementations, the filtering may include sources from other sensors to remove common mode or other noise and can use adaptive filtering techniques to remove unwanted signals. The filtered sensor data is transformed to enhance coughing components by modeling the input signal as a collection of waveforms of a particular form (sinusoids for the Fourier transform, mother wavelets for the wavelet transforms, periodic basis functions for the periodicity transforms) (703). In implementations, the transform can be a Fourier transform, wavelet transform, cosine transform or a math operation such as root-mean-square, absolute, moving average, moving median, etc. Envelope detection can be performed to take a relatively high-frequency amplitude modulated signal as input and provide an output which is equivalent to the outline of the input data described by connecting all the local peaks in this signal (704). In implementations, envelope detection can use a low pass filter, a Hilbert transform or other envelope detection methods. Patterns in the processed sensor data can be detected that match the morphology or spectral signature of coughing (705).

Variation analysis can be performed to measure the level of change in the data compared to be baseline (706). In implementations, this can be done by estimating a standard deviation, coefficient of variation, and the like. The variation analysis and cough signatures can be used to identify individual coughing episodes in the input signal (707). Time domain, frequency domain, or time frequency domain analysis can be used to determine the coughing rate (708). Time domain, frequency domain, or time frequency domain analysis can be used to determine the cough severity (709). Irregular coughs can be determined using the cough rate, cough severity, and subject's trend data 710 (i.e., baseline and historical data) (711).

The processing order shown in FIG. 7 is illustrative and the processing order may vary without departing from the scope of the specification or claims.

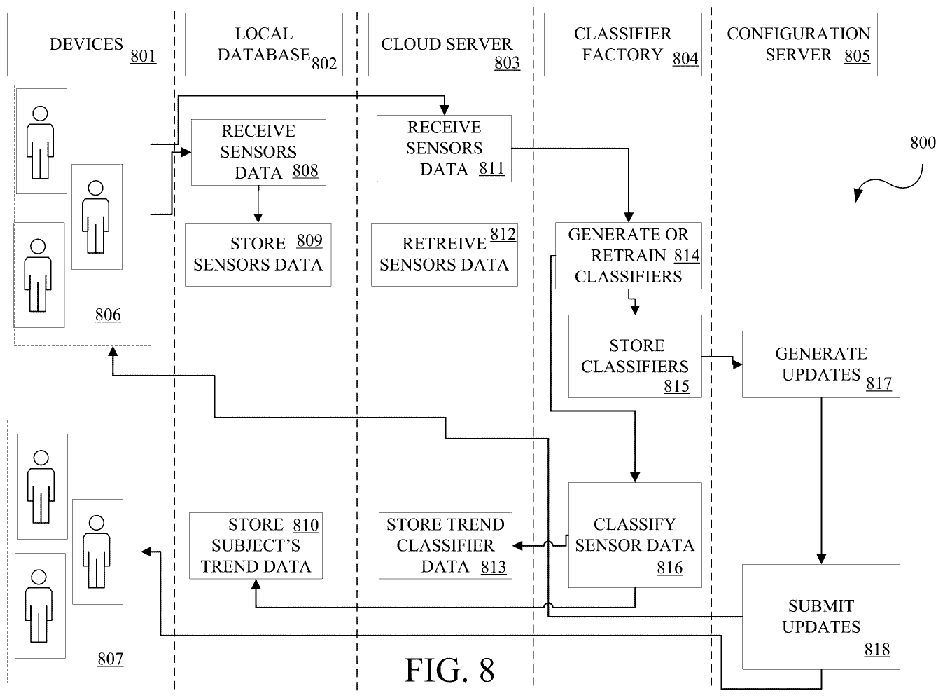

FIG. 8 is an example process for short-term screening and triage based on machine learning classifiers. A swim lane diagram 800 includes devices 801 which include a first set of devices 806 and a second set of devices 807, a local database 802, a cloud server 803, a classifier factory 804, and a configuration server 805.

The first set of devices 806 generate sensors data which are received (808) and stored (809) by the local database 802, and received by the cloud server 803. The cloud server 803 retrieves the sensor data (812) and the classifier factory 804 generates or retrains classifiers (814). The generated or retrained classifiers are stored by the classifier factory 804 (815). The generated or retrained classifiers are used by the classifier factory 804 to classify sensor data (816) and automatically detect different arrhythmias, diseases or out-of-the-norm conditions. The classified data is stored (813) and subjects trend data is stored (810). The configuration server 805 obtains the generated or retrained classifiers and generates an update for devices 801 (817). In implementations, the update can be an app update for the smart devices or a software update for remote devices. The configuration server 805 sends the update (818) to both the first set of devices 806 and to the second set of devices 807, where the second set of devices 807 may be new devices. The system can be used to provide new or updated classifiers to old devices (such as the first set of devices 806) as more data input is available from more devices. The system can also be used to provide software updates with improved accuracy and can also learn personalized patterns and increase personalization of classifiers or data.

The processing order shown in FIG. 8 is illustrative and the processing order may vary without departing from the scope of the specification or claims.

In general, a system for at least short term screening and triage of a subject includes a smart device in the subject's possession; an application provisioned on the smart device, the application and the smart device configured to provide instructions to the subject to start a screening procedure, the application programmed with multiple screening procedures, record short term sensor data obtained from one or more sensors located in the smart device, and compare the short term data, trend data, and general population data for screening and triage of the subject.

In implementations, the application and the smart device further configured to analyze the short term sensor data, obtain trend data from long term screening, identify conditions based on analyzed short term sensor data and trend data, and perform an action responsive to the identified conditions. In implementations, the action includes one or more of: generating an audible tone for a critical condition, sending a message to a cloud entity for a critical condition, sending the sensor data and identified conditions to an entity not the subject. In implementations, the application and the smart device further configured to initiate a long term screening to generate the trend data responsive to the identified conditions, wherein a long term screening system includes one or more sensors installed proximate to a substrate on which the subject is positioned, each sensor configured to capture mechanical vibrations from actions of the subject relative to the substrate, the mechanical vibrations indicative of biosignal information of the subject. In implementations, the long term screening system further configured to access the short term sensor data and identified conditions. In implementations, the application and the smart device further configured to initiate further short term screening responsive to the identified conditions. In implementations, the application and the smart device further configured to analyze cardiac information of the subject, determine cardiac rhythm and rate information from the cardiac information, determine the subject's health status from the cardiac rhythm and rate information, and identify onset or progression of a cardiac condition. In implementations, the application and the smart device further configured to: analyze respiratory information of the subject, determine respiration rhythm and rate information from the respiratory information, determine the subject's health status from the respiration rhythm and rate information, and identify onset or progression of a respiratory condition. In implementations, the application and the smart device further configured to: analyze coughing information of the subject, determine coughing rhythm and rate information from the coughing information, determine the subject's health status from the coughing rhythm and rate information, and identify onset or progression of a coughing condition or breathing flow. In implementations, the application and the smart device further configured to the application and the smart device further configured to determine the severity or progression of the condition. In implementations, the smart device is one of a cell phone, tablet, smart watch or accessory that has one or more of an accelerometer, gyroscope, pressure sensor, load sensor, weight sensor, force sensor, motion sensor, microphone, or vibration sensor. In implementations, the screening procedure includes instructions for the subject to get in a position, stay in the position for a defined time, and place the smart device at one or more positions on a body of the subject.

In general, a system for screening and triage of a subject includes a smart device provisioned with an application, collectively configured to: instruct a subject to start a screening procedure, record short term sensor data obtained from at least one sensor located in the smart device, and identify a condition from the short term sensor data and obtained trend data, a substrate deployed with sensors, the sensors configured to capture mechanical vibrations from actions of the subject relative to the substrate, the mechanical vibrations indicative of biosignal information of the subject, and a processor connected to the sensors, the processor configured to: capture sensor data from the sensors responsive to a smart device identified condition, identify a condition from the sensor data captured from the sensors and obtained short term sensor data, and perform an action based on an identified condition.

In implementations, the application and the smart device further configured to: analyze the short term sensor data sensor data, obtain trend data from storage associated with the processor, and perform an action responsive to the smart device identified condition. In implementations, the application and the smart device further configured to: transmit the short term sensor data sensor data to an entity for analysis against trend data, obtain results of analysis, and perform an action responsive to an entity identified condition. In implementations, the action responsive to the smart device identified condition or the action responsive to an entity identified condition includes one or more of: generating an audible tone for a critical condition, sending a message to a cloud entity for a critical condition, sending the short term sensor data and identified conditions to an entity not the subject. In implementations, the application and the smart device further configured to initiate further smart device screening responsive to the smart device identified condition. In implementations, the application and the smart device further configured to perform at least one of: analyze cardiac information of the subject, determine cardiac rhythm and rate information from the cardiac information, determine the subject's health status from the cardiac rhythm and rate information, and identify onset or progression of a cardiac condition, or analyze respiratory information of the subject, determine respiration rhythm and rate information from the respiratory information, determine the subject's health status from the respiration rhythm and rate information, and identify onset or progression of a respiratory condition, or analyze coughing information of the subject, determine coughing rhythm and rate information from the coughing information, determine the subject's health status from the coughing rhythm and rate information, and identify onset or progression of a coughing condition or breathing flow.

In general, a method for at least short term screening and triaging of a subject, the method includes instructing a subject, via a smart device, to initiate a screening procedure, recording, by a sensor on the smart device, short term sensor data from the subject as he/she follows the screening procedure, analyzing the short term sensor data, obtaining trend data from a long term screening device, identifying a subject condition based on analyzed short term sensor data and the trend data, and performing an action responsive to an identified condition.

In implementations, the method includes initiating capturing of sensor data at the long term screening device responsive to an identified condition. In implementations, the method includes analyzing long term screening device sensor data, obtaining smart device data, identifying a subject condition based on analyzed long term screening device sensor data, general population data, and the short term sensor data, and performing an action responsive to a long term screening device identified condition. In implementations, the method includes sending short term sensor data, smart device identified condition, long term screening device sensor data, and long term screening device identified condition, respectively, to at least an entity not the subject. In implementations, the method includes initiating additional smart device screening responsive to the identified condition.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

1-24. (canceled)

25. A system comprising:
a smart phone comprising:
a user interface comprising a display;
one or more sensors configured to monitor a heart activity of the user;
one or more processors and memory storing instructions configured to perform steps including:
driving the user interface to instruct the user to place the smart phone on an application-specified location of a body of the user;
receiving, from the one or more sensors, short term sensor data that represents the heart activity of the user;
comparing the short term sensor data that represents the heart activity with both trend data of the heart activity and general population data of persons other than the user; and
output a signal in response to comparing.

26. The system of claim 25, wherein the application-specified location of the body is a chest of the user.

27. The system of claim 25, wherein the instructions stored on the memory further include the step of driving the user interface to instruct the user to lie on a side of the user.

28. The system of claim 25, wherein the instructions stored on the memory further include the step of driving the user interface to instruct the user to get in a position and stay in the position for a defined time.

29. The system of claim 25, and further comprising:
a cloud server in data communication with the smart phone and configured to receive data from the smart phone; and
a classifier factory in data communication with the cloud server, wherein the classifier factory is configured to retrain classifiers and automatically detect different arrhythmias, diseases or out-of-the-norm conditions.

30. A system for at least short term screening and triage of a user, the system comprising:
a smart device wherein the smart device comprises:
a user interface configured to output instructions;
one or more sensors located in the smart device, wherein the one or more sensors are configured to monitor a heart activity of the user when the smart device is physically positioned on a chest of the user and proximate to a heart of the user;
one or more processors; and
storage associated with the one or more processors storing instructions configured to perform steps including:
driving the user interface to provide instructions to start a heart activity screening procedure that includes instructions to the user to lie down on an application-specified side of a body of the user and physically place the smart device on one or more application-specified locations on the body of the user;
receiving, from the one or more sensors, short term sensor data that represents a heart activity of the user during the heart activity screening procedure;
recording the short term sensor data obtained from the one or more sensors located in the smart device during the heart activity screening procedure;
accessing trend data that includes historical data of the heart activity prior to the heart activity screening procedure;
accessing general population data that includes historical data of heart activity of at least one or more individuals who are not the user;
comparing the short term data, the trend data, and the general population data for screening and triage of the user; and
outputting a signal in response to comparing the short term data, the trend data, and the general population data.

31. The system of claim 30, wherein the smart device is further configured to:
analyze the short term sensor data;
obtain the trend data from a long term screening that occurred prior to the heart activity screening procedure and for a duration longer than a length of the heart activity screening procedure;
identify a condition based on the trend data and analyzing the short term sensor data; and
output a signal to perform an action responsive to the identified condition.

32. The system of claim 31, wherein the action includes one or more of:
generating an audible tone;
sending a message to a cloud entity;
sending the short term sensor data to an entity who is not the user; or
sending the identified condition to the entity who is not the user.

33. The system of claim 31, wherein the smart device is further configured to:
initiate a long term screening system that generates the trend data, wherein the long term screening system includes at least one of the one or more sensors that are installed proximate to a substrate on which the user is positioned, each sensor configured to capture mechanical vibrations from actions of the user relative to the substrate, the mechanical vibrations indicative of biosignal information of the user.

34. The system of claim 31, wherein the smart device is further configured to:
initiate further short term screening responsive to the identified condition.

35. The system of claim 31, wherein the smart device is further configured to:
analyze cardiac information of the user that represents the heart activity;
determine both a heart rate and a heart rate variability from the cardiac information;
determine a health status for the user from both the heart rate and the heart rate variability; and
in response to identifying the condition, output a notification based on a property of the condition.

36. The system of claim 31, wherein the smart device is further configured to:
analyze respiratory information of the user;
determine a respiration rhythm and rate information from the analyzed respiratory information;
determine a health status for the user from the respiration rhythm and the rate information; and
identify an onset or a progression of a respiratory condition.

37. The system of claim 31, wherein the smart device is further configured to:
analyze coughing information of the user;
determine a coughing rhythm and rate information from the analyzed coughing information;
determine a health status for the user from the coughing rhythm and the rate information; and
identify an onset or a progression of a coughing condition or a breathing flow.

38. The system of claim 31, wherein the smart device is further configured to determine a progression of the condition.

39. The system of claim 30, wherein the smart device is one of a cell phone, tablet, smart watch or accessory and wherein the smart device comprises an accelerometer, a gyroscope, a pressure sensor, a load sensor, a weight sensor, a force sensor, a motion sensor, a microphone, or a vibration sensor.

40. The system of claim 30, wherein to provide the instructions to start the heart activity screening procedure, the smart device is configured to instruct a screen of the smart device to display the instructions to start the heart activity screening procedure.

41. The system of claim 30, wherein the application-specified side of the body is specified as at least one of the group consisting of a side, a back, a stomach, and an abdomen.

42. The system of claim 30, wherein the application-specified side of the body is specified as an abdomen.

43. The system of claim 30, wherein the one or more application-specified locations on the body are specified as at least one of the group consisting of a chest, an abdomen, a side, and a back.

44. The system of claim 30, wherein the one more application-specified locations on the body are specified as a back.

45. The system of claim 30, wherein the application-specified side of the body is specified as an abdomen and wherein the one more application-specified locations on the body are specified as a back.
